(19) United States
(12) Patent Application Publication
(10) Pub. No.: US 2018/0027853 A1
(43) Pub. Date: Feb. 1, 2018

(54) EMBRYONIC EGG EXTRACT COMPOSITION

(71) Applicant: Nitit Siripanich, Tarrytown, NY (US)

(72) Inventor: Nitit Siripanich, Tarrytown, NY (US)

(21) Appl. No.: 15/782,795

(22) Filed: Oct. 12, 2017

Related U.S. Application Data
(60) Provisional application No. 62/526,234, filed on Jun. 28, 2017.

(51) Int. Cl. A23L 15/00

(52) U.S. Cl. CPC A23L 15/00; A61K 9/20

(57) ABSTRACT

A composition of avian embryonic egg extract, panax Ginseng, and Ganoderma lingzhi extract is provided which may help to restore damaged cells, repair damaged body tissues, delay cellular deterioration, and inhibit free radicals,

### BACKGROUND OF THE INVENTION

1. Field of Invention

The present invention relates to the field of organic compositions for enhancing cellular performance.

2. Description of Related Art

In 1929, John R. Davidson, a Canadian Doctor, discovered an extract derived from fertilized avian eggs when they were at a critical stage of development. He used this extract to restore health in his patients. Dr. Davidson spent well over a decade developing and researching his theory. However, when Dr. Davidson passed away in 1943, his research on fertilized avian eggs was not passed on and was soon forgotten. Nearly 50 years later, the pursuit of fertilized avian egg extract was revived by Norway's foremost expert on egg research: Dr. Bjoedne Eskeland. He took Dr. Davidson's original research a step further and hypothesized that fertilized avian eggs contained a special combination of amino acids, peptides and protein fractions that could help provide an incredible array of health benefits when consumed by humans. This included vitamins, minerals and proteins, as well as important defense elements, growth factors, hormones and other biologically active components.

Based on the foregoing, there is a need in the art for an organic composition that improves cellular performance of the body's cells, that is non-toxic and easily accessible by those who desire it.

### SUMMARY OF THE INVENTION

An embryonic egg extract composition is provided consisting of an egg extract derived from a fertilized avian embryonic egg; an extract derived from panax Ginseng; and an extract derived from species selected from Ganoderma lucidum, Ganoderma tsugae and Ganoderma lingzhi.

The foregoing, and other features and advantages of the invention, will be apparent from the following, more particular description of the preferred embodiments of the invention, the accompanying drawings, and the claims.

### DETAILED DESCRIPTION OF PREFERRED EMBODIMENTS

The composition (referred to as High Density Extract or HDE™) is preferably formed of 1) fertilized avian early embryonic egg extract 2) Panax Ginseng Extract, and 3) Lingzhi Extract. The composition involves taking a nutrient from 9-day-old eggs in Scandinavia called Avian Embryonic eggs, extracting it with a high technology, Proto-Embryonic Stage Extract (PESE). This nutrient is rich in amino acid, glycopeptides and oligopeptides, which have small particles and thus can be easily absorbed into the body and used instantly. The oligopeptides from Avian Embryonic eggs, which exist in chicken eggs during the first 9 days, are densely rich in nutrients needed for cell nourishment, and are highest in hen eggs. Moreover, with the process of blending with extracts from Lingzhi mushroom from Japan and a special variety of Panax ginseng from Korea, the composition contains particular nutrients of all these extracts, amino acid, glycopeptides, oligopeptides and polysaccharide, with saponin and malphenol and subjected the mixture to the process of linking the structures of molecular density, which resulted in High Density Extract (HDE™). As this special nutrient enters the body, it will promote the functioning processes of the cells to reach the perfect condition and the highest efficiency. Thus, the cells throughout the body will be in a state of equilibrium and wellness. These processes also send energy to the brain and the mind, allowing the body to restore and repair itself to wellness, causing the immune system to generate the immune substance naturally, and, importantly, this enables the cells in the body to function more efficiently. At the same time, the nutrient protects cells from being destroyed, enhances the cell degeneration prevention system as well as delays cell degeneration, restores the functioning of the cells to the highest efficiency and repairs them, making them strong.

Fertilized avian early embryonic egg is the main component of the composition. The health benefits of the hen egg have been known for centuries. Investigation of the mechanism of the development of an egg after fertilization revealed certain health benefits. In earlier studies, whilst monitoring weight gain of the egg during their development, scientists found very little gain in the first 9-10 days (7.5%), and then a sharp increase (1190% by day 23), suggesting rapid development of a body. The potency of the nutrients available to the fertilized avian egg at this stage has always been assumed to be high, but it was only recently that the chemical structure of the original egg solids for these critical stages, termed blastodermal to protoembryoinic stages was obtained. During the blastodermal to protoembryonic stages of embryogenesis, oligopeptides with molecular weights from 0.5 to 1.0 kD were identified. Oligopeptides are compounds, which have 2 to 20 amino acids joined by a peptide bond. These short chains of amino acids are able to cross the digestive barrier without breaking down or changing the ratios and proportions. Peptides are far more potent than other neurotransmitters, requiring only small amounts to produce a profound effect.

Additionally, the uptake of the Fibroblast Growth Factor (FGF) (present in the protoembryonic fluid) by the developing avian egg sharply increases between days 11 & 12. These peptides and the FGF have been isolated through a proprietary process precisely at the right stage of development, using a proprietary drying technique to bring the health benefits to humans. The extract is termed Fertilized Avian Egg Extract (FAEE).

The bioactive peptides in FAEE stimulate the dormant stem cells to utilize the phyto amino acids and marine protein to repair damaged aged cells. Drying the protoembryonic fluid before the peptides are “used up” to build organs and bones, allows us to provide this building, repairing, maintenance mechanism of perfectly balanced amino acids, peptides and growth factors to humans.

Nature has devised an extremely versatile mechanism to provide nutrition with miraculous precision to the embryo of living creatures. The precise blend of oligopeptides may be seen as building blocks, without a bridge, or a director. The role of such a director is fulfilled by a growth factor known as the Fibroblast Growth Factor, or FGF, also a bioactive peptide. FGF is prolific in protoembryonic liquid as well as the human placenta. On the 11th day of the incubation cycle of a chicken egg, the chicken tissue shows a steep increase in these bioactive peptides, with the appropriate peptides to form the solid organs and bones. A detailed day-by-day study was performed in 1988. Discovered only in the seventies, FGF and bioactive peptides are critical in the development of embryos, including humans.

Bioactive peptides are responsible for building the linings in the blood vessels, creating the infrastructure for the nutrients to flow to critical areas of the brain and organs. Research credits bioactive peptides with the potential to directly affect many neuro disorders because of clear results of the ability of bioactive peptides to affect the growth of neurites (4). Neurites are signal senders (Axons) and signal receivers (dendrites) attached to the brain neurons.

Research (6) has also shown clearly that new cell cultures show a dramatic increase in peptide and amino acid uptake in the presence of FGF. This result gives credence to the hypothesis that embryonic growth is influenced by a very precise mechanism, which combines unique combinations of amino acids, peptides and FGF.

The beneficial impacts of FGF are: positive effects on memory, skin, libido, energy, joints, muscles, stress, sleep and emotional stability.

### Wound Healing Activity

In a 1997 study, immediately following surgery, (animal) subjects were randomized to receive either an amino acid diet or a peptide diet for 10 days and the strength of the wound was measured. Wound bursting pressure was found to be significantly higher in subjects receiving the peptide diets than in those just receiving amino acid diets. The authors suggest that dietary peptides may stimulate the production of growth factors such as growth hormone, insulin, or insulin growth factor (IGF-1). They also postulate that it is possible that the amino acid entry into the cell via peptide transporters is more efficient for stimulation of protein synthesis than entry in the form of just amino acids. Other possible mechanisms suggested by the authors for the increased wound healing with peptide versus non peptide diets include stimulation of collagen synthesis, increased blood flow to the wound, free radical scavenging, and generation of cytokine profiles which better support wound healing.

### Cortisol Study

This study was designed to ascertain the effect of the nutritional supplement, FGF on cortisol levels in the body. During the experiment, 28 subjects, 16 women and 12 men, between the ages of 36 and 83 took part in the study. Salivary cortisol level content of each participant was measured prior to him/her taking part in the study. This figure is known as “pre-FGF usage level.” The salivary cortisol level was also measured every fifth day three times throughout the study when each participant's intake amount was changed. Overall, study participants' cortisol levels were reduced by an average of 23.7 percent, where 16 started on a higher intake of FGF—four capsules, twice a day—and 12 started on one capsule twice per day. Participants that initially started on a higher intake of FGF saw their cortisol level reduced significantly over the first four days as compared to subjects that began the study with a lower usage amount. However, at the end of the study, there was a small, although insignificant, difference in favor of the high initial intake. The total cortisol reduction by the end of the study was 27.3 percent in women and 19.2 percent in men.

Panax Ginseng Extract may be taken by mouth to improve thinking, concentration, memory, Alzheimer's disease, work efficiency, physical stamina, preventing muscle damage from exercise, and athletic endurance. In some cases, Panax Ginseng is used to help individuals coping with stress and as a general tonic for improving well-being. Panax ginseng is also used for depression, anxiety, general fatigue and chronic fatigue syndrome (CFS), multiple sclerosis, for boosting the immune system, and for fighting particular infections in a lung disease called cystic fibrosis. These infections are caused by a bacterium named Pseudomonas. Panax ginseng has been used to treat breast cancer and prevent ovarian cancer, liver cancer, lung cancer, and skin cancer. Other uses include treatment of anemia, chronic bronchitis, swine flu, prediabetes and diabetes, inflammation of the stomach lining (gastritis), fever, hangover, chronic obstructive pulmonary disease (COPD), HIV/AIDS, fertility problems and sexual dysfunction in men, to increase sexual arousal in women, and asthma. For example, Ssme men apply Panax ginseng to the skin of the penis as part of a multi-ingredient product for treating early orgasm (premature ejaculation).

Panax ginseng is also used for bleeding disorders, loss of appetite, vomiting, intestinal problems, gallstones, bad breath, fibromyalgia, sleeping problems (insomnia), nerve pain, joint pain, dizziness, headache, hearing loss, convulsions, disorders of pregnancy and childbirth, hot flashes due to menopause, common cold and flu, heart failure, high blood pressure, quality of life, wrinkled skin, and to slow the aging process.

Panax ginseng contains many active substances. The substances thought to be most important are called ginsenosides or panaxosides. Ginsenosides is the term coined by Asian researchers, and the term panaxosides was chosen by early Russian researchers.

The Lingzhi Mushroom, being of the Genus Ganoderma and species Ganoderma lucidum, Ganoderma tsugae or Ganoderma lingzhi, produces a group of triterpenes, called ganoderic acids, which have a molecular structure similar to steroid hormones. It also contains other compounds often found in fungal materials, including polysaccharides (such as beta-glucan), coumarin, mannitol, and alkaloids. Sterols isolated from the mushroom include, ganoderol, ganoderenic acid, ganoderiol, ganodermanontriol, lucidadiol, and ganodermadiol. Fungal immunomodulatory proteins (FIPs) are bioactive ingredients within genera Ganoderma that have immune building properties. FIPs stimulate different cells and cellular components that enable immune response. Some of the molecules and cells that FIPs influence include T and B lymphocytes, natural killer cells, macrophages, and regulation of human monocyte DCs. Improved function of these cells will promote cytokine expression such as IL2, IL-4, and IFN-gamma which will support lymphocyte proliferation, immune cell initiation, and tumor inhibiting factors. Immunostimulation from FIPs could cause increased expression of costimulatory molecules and major histocompatibility complexes (MHC) which help to create a pathway for immune response to take place.

Ganoderma lucidum is widely used and recommended by Asian physicians and naturopaths for its supporting effects on the immune system. Preliminary laboratory research and limited preclinical trials have suggested that G. lucidum has potential anticancer and immunomodulatory properties, and cancer patients have been using G. lucidum-derived supplements more commonly as an alternative medicine.

In one formulation, for particular potency and effect, doses of of 450 mg of avian early embryonic egg extract are combined with 50 mg of Panax Ginseng Extract and 35 mg of Lingzhi Extract. Therefore, the preferred ratio is 90:10:7 for the components.

The benefit of the composition includes restoring and enhancing cells. Our cells constantly deteriorate in daily life. If there is no support to restore the deterioration, cellular damage will affect the body functions. HDE™ composition is the supplementary factor that can restore the cells to the body balance without drug and substance usage. When cells are restored, in the next process, HDE™ composition will accordingly boost cell strength, equivalent to doubling the effectiveness and the number of cells, while maximizing physical and mental abilities After the cellular restoration and boosting the cell strength, the next challenge is the protection to maintain the long-lasting quality of the cells. HDE™ dietary supplement will help to protect and delay the cellular deterioration. This will also result in improving the immune system. When the cell functions are systematically restored, boosted and protected, HDE™ dietary supplement will directly enhance physical and mental capacity to go beyond limits at each age and to live life fully in the world of change.

The health benefits of HDE are also of particular value to Children, and help to develop the strength of new cells and protect cells from early deterioration. Both are beneficial to the development of body, intelligence, memory, and positive emotions, suitable for the colorful age ready to create their world. The Physical Results may include i) restore cells for normal growth in each stage, ii) recover from illness, iii) keep growth hormone function at an optimum level, iv) build muscle, v) boost the immune system, vi) reduce the risk of the incidence of disease, vii) promote growth to maximum effectiveness, viii) adjust height, ix) Improve body growth, x) Efficient immunity and xi) Increase Energy

The Mental Results may include: developing emotions and thoughts; relieving depression symptoms; improving attention deficit disorder symptoms; boosting intelligence and memory; reducing the risk of developmental delay; enhancing emotional Intelligence

The composition also helps to restore the old damaged cells, repair the damaged body tissues, delay the cellular deterioration, inhibit free radical, and help the process of skin cell renewal in adults for the glowing skin and younger look, Meanwhile, it also enhances the balance, reduces exhaustion from stress, improves the concentration and work efficiency, including boosting the immune system to stay healthy

Physical Results may include: a radiant and vibrant skin, younger look; reducing fatigue at work; restoring the reproductive system; refreshing the body; boosting the immune system; protecting against free radical formation; preventing premature aging

Mental Results may include restoring hormonal balance; boosting functions of sensory nervous system and memory.

The compounds are dried and ground to small particles, and are subsequently combined in the desired ratio. The combination may remain as a powder to be added to food or pressed in a tablet. Alternatively, the compounds may be mixed in an aqueous solution and provided in liquid form.

Therefore, in a preferred dosage, the total compound blended is 535 mg, pressed into a single, large tablet. The compound may be sold under the HDE name.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claim

I claim:

1. An embryonic egg extract composition consisting of:
an egg extract derived from a fertilized avian embryonic egg;
an extract derived from panax Ginseng; and
an extract derived from species selected from the group consisting of Ganoderma lucidum, Ganoderma tsugae and Ganoderma lingzhi.

2. The composition of claim 1, wherein the composition ratio of the fertilized avian embryonic egg extract, panax Ginseng extract, and Ganoderma lingzhi extract is about 90:10:7, by weight, respectively.

3. The embryonic egg extract composition of claim 1 wherein the embryonic egg is no more than 9 days old.

4. The embryonic egg extract composition of claim 1 consisting of 450 mg of avian embryonic egg extract, 50 mg of panax Ginseng extract and 35 mg of Ganoderma lingzhi extract.

5. The embryonic egg extract of claim 4 wherein the composition is provided in an aqueous solution.

6. The embryonic egg extract of claim 4 wherein the composition is provided in a tablet form.
